(19) United States
(12) Patent Application Publication
(10) Pub. No.: US 2019/0119694 A1
(43) Pub. Date: Apr. 25, 2019

(54) ENHANCED CANNABIS PLANTS AND METHODS OF MAKING AND USING THE SAME

(71) Applicant: ebbu, INC., Evergreen, CO (US)

(72) Inventor: Robert Fletcher ROSCOW, Jr., Boulder, CO (US)

(21) Appl. No.: 16/093,066

(22) PCT Filed: Apr. 14, 2017
(86) PCT No.: PCT/US2017/027643

§ 371 Date: Oct. 11, 2018

Related U.S. Application Data
(60) Provisional application No. 62/322,736, filed on Apr. 14, 2016.

(51) Int. Cl. C12N 15/82; A01H 6/28; A61K 31/352

(52) U.S. Cl. CPC C12N 15/8243; A61K 31/352; A01H 6/28

(57) ABSTRACT

Disclosed herein is a plant of the genus cannabis that does not require flowering in order to produce trichomes comprising secondary compounds. Provided herein is a plant of the genus cannabis that has trichomes on non-flowering parts of the plant, such as leaves. The disclosed plants have a high mass % of secondary compounds and a high degree of trichome coverage on the surface of the plant. Also disclosed herein are methods of producing secondary compounds from a plant of genus cannabis without flowering the plant of genus cannabis. For example, the disclosed methods provide for inducing trichome development on a plant of genus cannabis without flowering the plant of genus cannabis.

### CROSS REFERENCE TO RELATED APPLICATIONS

This application claims priority on the basis of U.S. Provisional Application No. 62/322,736 filed Apr. 14, 2016, which is hereby incorporated into reference.

### BACKGROUND

Cannabis is a genus of flowering plant. Plants of genus cannabis include three different species: Cannabis sativa, Cannabis indica, and Cannabis ruderalis. Plants of genus cannabis have long been used for hemp fiber, for seed and seed oils, for medicinal purposes, and for psychoactive properties.

Cannabis is composed of at least 483 known chemical compounds, which include cannabinoids, terpenoids, flavonoids, nitrogenous compounds, amino acids, proteins, glycoproteins, enzymes, sugars and related compounds, hydrocarbons, simple alcohols, aldehydes, ketones, simple acids, fatty acids, simple esters, lactones, steroids, terpenes, non-cannabinoid phenols, vitamins, pigments, and elements. These compounds are secreted on the glandular trichomes. Cannabinoids are unique to the cannabis plant and there have been 100 cannabinoids that have been isolated as purified (single) molecules.

Most extraction processes aim to extract cannabinoids from the flowering parts of the cannabis plant, particularly tetrahydrocannabinol (THC). THC has many effects including relieving pain, treating glaucoma, relieving nausea, and inducing vomiting during cancer treatments. The latter is sold as the drug dronabinol, a pure isomer of THC, (-)-trans-Δ9-tetrahydrocannabinol which is man made. The brand name in the US is Marinol.

Preparative gas chromatography of flower extracts has proven to be a suitable method for providing adequate pure samples of THC, Cannabidiol (CBD), and Cannabinol (CBN). There has been difficulty in isolating pure cannabinoids in order to conduct research. Cannabinoids can have synergistic or antagonistic effects on each other. Other methods of extraction include butane hash oil (BHO) and supercritical carbon dioxide extraction. Cannabinoids are drawn out of the plant through solvent (e.g., butane or carbon dioxide) extraction, which produces a purified composition.

The flowering parts of the cannabis plant include trichomes, which comprise the majority of the plant's secondary compounds, e.g., cannabinoids and terpenes. Trichomes can be separated from the plant by placing the whole plant in a fine mesh screen shifter and gently shaking so that the trichomes fall through the screen away from the plant. The crude trichomes are sometimes compressed into rounds known as hash or hashish.

Harvesting secondary compounds, e.g., cannabinoids and terpenes from a plant of the genus cannabis requires harvesting trichomes. Harvesting trichomes requires flowering a plant of the genus cannabis. From start to finish, harvesting secondary compounds from the trichomes of a plant of genus cannabis requires five stages of plant growth: Germination; Seeding; Vegetative Growth; Pre-Flowering; and Flowering.

Germination is the stage of plant growth, during which the seeds sprout and the root emerges. In Cannabis, it takes from 12 hours to 8 days for a seed to germinate. Warmth, darkness, and moisture initiate metabolic processes such as the activation of hormones triggering the expansion of the embryo within the seed. Then, the seed coat cracks open and a small embryonic root emerges and begins growing downward. After about 2-4 days, the root becomes anchored and two circular embryonic leaves (aka cotyledons) emerge in search of light, pushing the remains of the seed shell away. This marks the beginning of the seedling stage.

Germination can be initiated by soaking seeds either between wet paper towels, in a cup of water at room temperature, in wet peat pellets, or directly in potting soil. Peat pellets are often used as a germinating medium because the saturated pellets with their seedlings can be planted directly into the intended growing medium with a minimum of shock to the plant.

The seedling stage begins when the seed coat splits open and exposes the root and round “seed leaves” or cotyledons. The seedling stage lasts from 1 to 4 weeks and is the period of greatest vulnerability in the life cycle of the plant, requiring moderate humidity levels, medium to high light intensity, and adequate but not excessive soil moisture. A growing cannabis plant will naturally begin to develop identifiable sex characteristics during the seedling stage, after 4 to 6 weeks.

The plant of genus cannabis enters the vegetative stage after it develops seven sets of true leaves and the 8th is barely visible in the center of the growth tip. During the vegetative phase, the plant directs its energy resources primarily to the growth of leaves, stems, and roots. A strong root system is required for strong floral development. As a rule of thumb, a plant of genus cannabis needs about 1 to 2 months of vegetative growth before beginning the pre-flowering and flowering stages.

The pre-flowering stage, also called “the stretch,” begins when the photoperiod of the plant switches to 12 or more hours of darkness per 24 hours. The pre-flowering stage may last from one day to two weeks. Most plants spend 10-14 days in this period after switching the light cycle to 12 hours of darkness. During the pre-flowering stage, plant development increases dramatically. The plant may increase in size by 200+%. During the pre-flowering stage, the plant develops more branches and nodes. The plant structures required for flowering development. The plant starts to develop bracts/bracteoles where the branches meet the stem (nodes). Pre-flowering indicates the plant is ready to flower.

The flowering stage varies from about 6 to 22 weeks, depending on the type of cannabis plant. Cannabis indica plants are generally believed to require shorter flowering times that Cannabis sativa plants. During flowering, unpollinated female plants produce buds that contain sticky white resin glands or trichomes. These trichomes produce resins that contain the largest amounts secondary compounds, such as cannabinoids, like THC and CBN, and terpenes.

A variety of growing and cultivation techniques have been developed for harvesting secondary compounds from plants of genus cannabis. These techniques include outdoor cultivation, indoor cultivation, hydroponics, fertilizing, atmospheric manipulation, cloning, cross breeding, SCROG, SOG, pinching, training, topping, etc. All cultivation techniques share one common attribute: flowering a plant of genus cannabis in order to produce trichomes comprising the desired secondary compounds.

There exists a need for a plant of genus cannabis that does not require flowering in order to produce trichomes comprising secondary compounds.

There exists a need for a plant of genus cannabis that has trichomes on non-flowering parts of the plant, such as leaves.

There exists a need for a plant of genus cannabis comprising a high mass % of secondary compounds.

There exists a need for methods of producing secondary compounds from a plant of genus cannabis without flowering the plant of genus cannabis.

There exists a need for methods of inducing trichome development on a plant of genus cannabis without flowering the plant of genus cannabis.

### DETAILED DESCRIPTION

Disclosed herein is a plant of the genus cannabis that does not require flowering in order to produce trichomes comprising secondary compounds.

Disclosed herein is a plant of the genus cannabis that has trichomes on non-flowering parts of the plant, such as leaves.

Disclosed herein is a plant of the genus cannabis comprising a high mass % of secondary compounds.

Disclosed herein are methods of producing secondary compounds from a plant of genus cannabis without flowering the plant of genus cannabis.

Disclosed herein are methods of inducing trichome development on a plant of genus cannabis without flowering the plant of genus cannabis.

Disclosed herein is a plant of genus cannabis, having a surface area including trichomes on non-flowering parts of the plant. In some embodiments, the plant of genus cannabis has trichomes on 25-100% of the surface area of the plant. In some embodiments, the plant of genus cannabis has trichomes on 50-100% of the surface area of the plant. In some embodiments, the plant of genus cannabis has trichomes on 70-100% of the surface area of the plant.

Within the context of this disclosure, the “surface area of the plant” refers to the parts of the plant that are above the ground, not including the root system.

As used herein, the term “plant” means a multicellular eukaryote of the kingdom Plantae, whether naturally occurring, completely manmade, or some combination thereof.

As used herein, the term “plant of genus cannabis” means a plant belonging to the genus “cannabis” within the accepted biological taxonomical system, including the species Cannabis sativa, Cannabis indica, and Cannabis ruderalis.

As used herein, the term “surface area” means the total area that the surface of an object occupies. As used herein, the “surface area” of an object can be calculated at various degrees of precision or accuracy. Within the context of this disclosure, referring to “trichomes on” a stated percentage “of the surface area of the plant” refers to the percentage of the plant's exterior surface that is occupied by (or covered with) trichomes.

In some embodiments, the plant disclosed herein has 20-80 mass % secondary compounds. In some embodiments, the plant has 30-70 mass % secondary compounds. In some embodiments, the plant has 40-65 mass % secondary compounds. In some embodiments, the plant has 30-60 mass % secondary compounds.

As used herein, the term “mass % of secondary compounds” refers to the percentage of the plant's total mass that is comprised by secondary compounds. For example, a plant having a total mass of 1 kilogram, comprising 500 total grams of secondary compounds, would have 50 mass % of secondary compounds.

Disclosed herein is a plasmid that includes validated native cannabis cDNA fragments corresponding to trichome induction.

As used herein, the term “plasmid” refers to a small DNA molecule within a cell that is physically separated from a chromosomal DNA and can replicate independently. In one example the plasmid is pRI-201AN.

In one embodiment, the term “validated,” within the context of the cDNA within a plasmid, means that the cDNA sequence was confirmed via a technique such as restriction enzyme digest and Sanger sequencing.

As used herein, the term “cDNA” refers to complementary DNA, which is double-stranded DNA synthesized from a messenger RNA (mRNA) template in a reaction typically catalyzed by the enzyme reverse transcriptase. Within the context of this disclosure, the term “cDNA” may refer to naturally occurring, modified, or synthetic cDNA or combinations thereof in any proportion.

As used herein, the term “trichome induction” means bringing about the development of trichomes. Within the context of this disclosure, the term “trichome induction” may refer to promoting biological processes for making or growing trichomes. The term “trichome induction” may also refer to thwarting biological processes that decrease trichome production, such as interfering with a repressor of trichome development.

Disclosed herein is a purified transformed bacteria comprising native cannabis DNA. In one embodiment, the native cannabis DNA corresponds to trichome induction in a native cannabis plant.

As used herein, the term “transformed bacteria” refers to a genetically altered bacteria resulting from the uptake and incorporation of exogenous genetic material, e.g., DNA. Within the context of this disclosure, the term “purified transformed bacteria” refers to a transformed bacteria, which has been isolated from its natural surroundings. For example, a bacteria may be harvested from a growth medium by centrifugation. In some embodiments, the purified transformed bacteria is suspended in a buffer solution.

As used herein, the term “native cannabis DNA” means DNA found within a naturally occurring plant of the genus cannabis. Disclosed herein is a genetically modified Agrobacterium tumifaciens bacteria, comprising native cannabis DNA corresponding to trichome induction in a native cannabis plant.

In some embodiments, a plant of the genus cannabis has cDNA fragments corresponding to trichome induction in a plant not of genus cannabis.

As used herein, the term “plant not of genus cannabis” means a plant that does not belong to the genus “cannabis” within the accepted biological taxonomical system. For example a “plant not of genus cannabis” includes a plant of genus Arabidopsis. But, a “plant not of genus cannabis” does not include a plant chosen from Cannabis sativa, Cannabis indica, and Cannabis ruderalis.

Disclosed herein is a method of producing secondary compounds in a plant of genus cannabis, comprising inducing trichome development in a plant of genus cannabis. In some embodiments, the secondary compounds are chosen from cannabinoids or terpenes.

As used herein, the term “terpene” means an organic compound built on an isoprenoid structural scaffold or produced by combining isoprene units. Often, terpene molecules found in plants may produce smell.

The structure of terpenes are built with isoprene units, which are 5 carbon structures. Flavonoids are generally considered to be 15 carbon structures with two phenyl rings and a heterocyclic ring. So, there could be an overlap in which a flavonoid could be considered a terpene. However, not all terpenes could be considered flavonoids.

Within the context of this disclosure, the term terpene includes Hemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives.

Derivatives of terpenes include terpenoids in their forms of hemiterpenoids, monoterpenoids, sesquiterpenoids, sesterterpenoid, sesquarterpenoids, tetraterpenoids, Triterpenoids, tetraterpenoids, Polyterpenoids, isoprenoids, and steroids. They may be forms: α-, β-, γ-, oxo-, isomers, or combinations thereof.

Examples of terpenes within the context of this disclosure include: 7,8-dihydroionone, Acetanisole, Acetic Acid, Acetyl Cedrene, Anethole, Anisole, Benzaldehyde, Bergamotene (α-cis-Bergamotene) (α-trans-Bergamotene), Bisabolol (β-Bisabolol), Borneol, Bornyl Acetate, Butanoic/Butyric Acid, Cadinene (α-Cadinene) (γ-Cadinene), Cafestol, Caffeic acid, Camphene, Camphor, Capsaicin, Carene (A-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (α-Cedrene Epoxide), Cedrol, Cembrene, Chlorogenic Acid, Cinnamaldehyde (α-amyl-Cinnamaldehyde) (α-hexyl-Cinnamaldehyde), Cinnamic Acid, Cinnamyl Alcohol, Citronellal, Citronellol, Cryptone, Curcumene (α-Curcumene) (γ-Curcumene), Decanal, Dehydrovomifoliol, Diallyl Disulfide, Dihydroactinidiolide, Dimethyl Disulfide, Eicosane/Icosane, Elemene (8-Elemene), Estragole, Ethyl acetate, Ethyl Cinnamate, Ethyl maltol, Eucalyptol/1,8-Cineole, Eudesmol (α-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1(10),11-diene, Guaiacol, Guaiene (α-Guaiene), Gurjunene (α-Gurjunene), Herniarin, Hexanaldehyde, Hexanoic Acid, Humulene (α-Humulene) (β-Humulene), Ionol (3-oxo-α-ionol) (13-Ionol), Ionone (α-Ionone) (β-Ionone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (p-Myrcene), γ-Muurolene, Nepetalactone, Nerol, Nerolidol, Neryl acetate, Nonanaldehyde, Nonanoic Acid, Ocimene, Octanel, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, α-Sinensal, β-Sinensal, β-Sitosterol, Squalene, Taxadiene, Terpin hydrate, Terpineol, Terpine-4-ol, α-Terpinene, γ-Terpinene, Terpinolene, Thiophenol, Thujone, Thymol, α-Tocopherol, Tonka Undecanone, Undecanal, Valeraldehyde/Pentanal, Verdoxan, α-Ylangene, Umbelliferone, or Vanillin.

As used herein, the term “cannabinoid” means any substance that acts upon a cannabinoid receptor. For example the term cannabinoid includes cannabinoid ligands such as agonists, partial agonists, inverse agonists, or antagonists, as demonstrated by binding studies and functional assays. In many examples, a cannabinoid can be identified because its chemical name will include the text string “*cannabi* in the name. Within the context of this application, where reference is made to a particular cannabinoid, each of the acid and/or decarboxylated forms are contemplated as both single molecules and mixtures.

Examples of cannabinoids within the context of this disclosure include compounds belonging to any of the following classes of molecules, their derivatives, salts, or analogs: Tetrahydrocannabinol (THC), Tetrahydrocannabivarin(THCV), Cannabichromene(CBC), Cannabichromanon (CBCN), Cannabidiol (CBD), Cannabielsoin (CBE), Cannabidivarin (CBDV), Cannbifuran (CBF), Cannabigerol (CBG), Cannabicyclol (CBL), Cannabinol (CBN), Cannabinodiol (CBND), Cannabitriol (CBT), Cannabivarin (CBV), and Isocanabinoids.

Disclosed herein is a method of harvesting at least one cannabinoid or at least one terpene during the vegetative growth cycle of the plant of the genus cannabis.

As used herein, the phrase “harvesting . . . during the vegetative growth cycle” means collecting secondary compounds while the plant is within the vegetative stage of growth as opposed to waiting until the plant flowers.

In some embodiments, the method of harvesting at least one cannabinoid or at least one terpene during the vegetative growth cycle of the plant of the genus cannabis includes modifying genetic material of the plant of the genus cannabis.

In one embodiment, “modifying genetic material of the plant of the genus cannabis” includes independently overexpressing one or more single genes that induce trichrome development. In one embodiment, one or more genes are chosen from available literature, and isolated from the closest relative with published sequence data. The isolated DNA was inserted into an expression cassette. Overexpression of mRNA was accomplished via a CaMV 35S promoter sequence. Robust protein expression was accomplished with AtADH 5′ UTR and HSP 3′ UTR sequences. This expression cassette was inserted into the target Cannabis genera plant genome using a binary vector Agrobacterium mediated system. Small-scale transgenesis was accomplished at a local scale with syringe infiltration, and in the whole plant via vacuum infiltration.

In one embodiment, “modifying genetic material of the plant of the genus cannabis” includes target gene expression. In one embodiment, target gene expression comprises underexpressing at least one trichome induction/patterning gene. In one embodiment, one or more genes are chosen from available literature, and isolated from the closest relative with published sequence data.

In some embodiments, the method of harvesting at least one cannabinoid or at least one terpene during the vegetative growth cycle of the plant of the genus cannabis includes introducing non-native DNA to the plant of genus cannabis.

In some embodiments, the method of harvesting at least one cannabinoid or at least one terpene during the vegetative growth cycle of the plant of genus cannabis includes introducing additional copies DNA native to the plant of genus cannabis.

In some embodiments, the method of harvesting at least one cannabinoid or at least one terpene during the vegetative growth cycle of the plant of genus cannabis includes overexpressing at least one trichome induction gene. In some embodiments, at least one trichome induction gene is chosen from the bHLH, WD40 repeat protein, R2R3-MYB, or R3-MYB families.

In some embodiments, the method of harvesting at least one cannabinoid or at least one terpene during the vegetative growth cycle of the plant of the genus cannabis includes infecting cells of the plant of genus cannabis with a transformed bacterium. In some embodiments, the method includes infecting cells of the plant of genus cannabis with a transformed bacterium via syringe infiltration. In some embodiments, the method includes infecting cells of the plant of genus cannabis with a transformed bacterium via vacuum infiltration.

In one embodiment, “syringe infiltration” was accomplished as follows: 10 ml or 50 ml syringe without a needle and loaded with a bacteria solution. The syringe tip was place flat against the underside of the leaf. A gloved finger was placed against the top of the leaf to apply pressure. The plunger was slightly depressed, allowing the fluid to travel through the open stomata into the intercellular space in the leaf, where the bacteria can infect the cells and subsequently insert our expression cassette with the gene of interest. This procedure was carried out when the plants were under bright lighting to ensure active transpiration and open stomata. Plants were grown and assessed for expression at the injection site. Localized transgenesis was observed.

In one embodiment, “vacuum infiltration” was accomplished as follows: Small rooted clones were suspended upside down in a bath of bacteria solution; all leaves, stems and growth tips were submerged and the roots left exposed; the bath was situated in a vacuum chamber and a vacuum was applied to the roots, thereby pulling the solution into the leaves through the open stomata and the rest of the plant via the vascular system. This procedure resulted in mosaic expression of the transgene in the whole plant. The plant was then grown and sub cloned using traditional plant cloning and/or plant tissue culture. In one embodiment, the sub-cloning step comprises using an antibiotic. The clone was usable in a normal cannabis cultivation scenario, while expressing the transgene and it's resulting phenotype.

[60] In some embodiments, the method of harvesting at least one cannabinoid or at least one terpene during the vegetative growth cycle of the plant of the genus cannabis includes treating cells of plants of genus cannabis with DNA attached to a metal particle via biolistic particle delivery.

In some embodiments, the method of harvesting at least one cannabinoid or at least one terpene during the vegetative growth cycle of the plant of genus cannabis includes repressing post-transcriptional processing of a transcriptional repressor of trichome induction and/or repressing expression of a target gene as a functional protein.

In some embodiments, the method of harvesting at least one cannabinoid or at least one terpene during the vegetative growth cycle of the plant of genus cannabis includes expressing an RNA interference molecule corresponding to a trichome induction repression gene and/or reducing translation of mRNA trichome induction repression genes into functional protein.

In some embodiments, the method of harvesting at least one cannabinoid or at least one terpene during the vegetative growth cycle of the plant of the genus cannabis includes modifying expression of GLABROUS INFLORESENCE STEMS (GIS), GLABROUS INFLORESENCE STEMS 2 (GIS2, GLABORUS INFLORESCENCE STEMS 3, (GIS3)COTYLEDON TRICHOME 1 (COT1), MYB5, ARPC5, MYB106, FZR2, HOMEODOMAIN GLABROUS 12(HDG12), BRANCHLESS TRICHOMES (BTL), HOMEODOMAIN GLABROUS 11 (HDG11), STICHEL (STI), IRREGULAR TRICHOME BRANCH1 (ITB1), KAKTUS (KAK), MYOSIN XI K, ATXIK, DYNAMIN-RELATED PROTEIN (DRP1A), ATRLCK VI_A3, ZWICHEL, FRC1, FRC3, FRC4, ITB2, ITB3, ITB4, RASTIFARI (RFI), STA, SUZ1, SUZ2, SUZ3, ZINC FINGER PROTEIN 5, ZINC FINGRE PROTEIN 6, ZINC FINGER PROTEIN 8, GL2-EXPRESSION MODULATOR (GEM), HARLEQUIN (HQL), TRANSPARENT TESTA 8 (TT8) in a plant of genus cannabis.

In some embodiments, the method of harvesting at least one cannabinoid or at least one terpene during the vegetative growth cycle of the plant of genus cannabis includes modifying expression of a gene chosen from the R2R3-MYB, bHLH, WD40 repeat protein, or R3-MYB gene families.

In some embodiments, the method of harvesting at least one cannabinoid or at least one terpene during the vegetative growth cycle of the plant of genus cannabis includes physically damaging the DNA corresponding to a transcriptional repressor of trichome induction.

### EXAMPLES

Below are several provided examples intended to illustrated specific embodiments of this disclosure. These examples are illustrative and not intended to be limiting of the broader scope of the disclosure.

### Example 1

### Illustration of cDNA Creation

Arabidopsis thaliana seeds (genotype: Col-1) were germinated and grown for 48 and 72 hours saturated in diH2O at room temperature. Sprouted seeds were harvested into micro-centrifuge tubes and immediately frozen at −80 deg. Centigrade for storage prior to nucleic acid extraction. Samples from both 48 and 72 hours were pooled and their RNA extracted using a standard commercial kit and protocol (Qiagen, RNeasy Plant Mini Kit).

### Example 2

### Creation of cDNA Library

The total RNA extract described in Example 1 was used as a template for creation of a cDNA library through reverse transcription. The reaction was performed with a reverse transcriptase (SuperScript IV), and priming using random hexameter oligos.

### Example 3

### DNA Construct Assembly and Bacterial Culture

To express the target gene EGL3 from Arabidopsis thaliana (AtEGL3) in cannabis, a ubiquitous protein overexpression construct was utilized.

First, primers specific to amplify the entire mRNA sequence for the AtEGL3 gene were designed based on available sequence information. The target gene was amplified by using the Polymerase Chain Reaction.

### Example 4

### Generation of Single Colony Transformants

The product described in Example 3 was sequenced, verified, and used with the vector plasmid. The vector plasmid, contains a CaMV35S plant promoter sequence, a 5&#39; UTR sequence from the Arabidopsis thaliana Alcohol Dehydrogenase Gene, and a 3&#39; UTR sequence from a Arabidopsis thaliana heat shock protein gene. The AtEGL3 gene was ligated into the vector plasmid between the above described UTRs and in-frame with the transcription start site present in the AtADH 5&#39; UTR.

The above described plasmid was then cloned into E. coli competent cells. Single colonies were then screened for a correctly assembled plasmid. A sample of the plasmid from the screened colony was purified and subsequently transformed into Agrobacterium tumefaciens. Single colonies of these Agrobacterium transformants were grown in small liquid culture and used to make frozen cell bullets for later use in plants.

### Example 5

### Agrobacterium Inoculation

Multiple identical iterations of the following protocol were performed in parallel to test different transformed strains of Agrobacterium from the previous description. This description as follows traces one of these exemplary replicates.

A scraping from a single cell bullet of transformed Agrobacterium, described in Example 4, was used to inoculate a bacterial media plate and then incubated. A single colony from this plant was used to inoculate a large (0.1-1L) liquid culture. This culture was incubated by shaking until growth had reached log stage. Cells were then harvested via centrifugation and re-suspended in infiltration media supplemented with MES and Acetosyringone.

### Example 6

### Plant Transformation

Local Scale: The plant inoculation solution described in Example 5 was injected into the interstitial leaf of growing Cannabis plant fan leafs. This was accomplished with a 25 ml wide bore syringe. The fan leaf was turned upside down and pressure was applied to the top surface directly opposed to the syringe barrel tip on the bottom of the leaf. By applying light pressure, inoculation fluid was injected into an area of the leaf measuring approximately 2 inches by 2 inches. All leaves were tagged and scored one week later for trichome production at the injection site. Plants were maintained in normal intensive cultivation conditions throughout the process.

### Example 7

### Whole Plant Production and Analysis

The inoculation solution described above was placed in a beaker and a small cannabis plant was suspended and inverted in the solution with the roots above the liquid level. This resulting setup was then placed into a vacuum chamber and exposed to a short duration of reduced pressure, thereby pulling the solution into the plant. After this treatment, the plant was grown under normal conditions for 2 weeks. Trimmings were subsequently taken from this treated plant and grown. The plants grown from these cuttings were assessed for trichome production throughout growth.

### Example 8

### Observing Over Expression of One bHLH Gene, AtEGL3 in Cannabis

Local Scale: Most injection sites on fan leaves of overexpressed plants showed some trichome formation whereas the fan leaves of natural plants showed only a negligible amount of trichomes.

The phenotype of ectopic trichomes observed growing from the injection site and close surrounding leaf tissue was starkly visually apparent. Analysis of digital images taken of the fan leaves for trichome density surrounding the injection site showed an increase in trichome density: an average of 8.4 times the number of trichomes near an injection site when compared to a non-injected blade of the same leaf

### Example 9

### Whole Plants

Plants made with the method described above were grown and flowered following standard cannabis cultivation methods. Unmodified plants from the same mother as the modified ones were also grown in the same environment. The THC production of these plants were measured at the end of the flowering cycle.

Results for the THC production of the flowers tested within this population demonstrated significant variability between the modified versus unmodified plants. Fan leaves on the modified plants displayed a variable phenotype with some covered in trichomes and some not. Of the ones that were trichome covered, their THC content was 10.6% compared to naturally grown plants (not subject to an overexpressing protocol), which were measured to have THC content of less than 1%. The modified plants displayed trichomes earlier than the un-modified comparators during growth. The modified plants produced significantly more purple pigmentation than the unmodified plants.

Although the present invention herein has been described with reference to various exemplary embodiments, it is to be understood that these embodiments are merely illustrative of the principles and applications of the present invention. Those having skill in the art would recognize that various modifications to the exemplary embodiments might be made, without departing from the scope of the invention.

Moreover, it should be understood that various features and/or characteristics of differing embodiments herein might be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s).

What is claimed is:

1. A plant of genus cannabis, having a surface area and comprising trichomes on non-flowering parts of the plant.

2. The plant of claim 1 comprising trichomes on 25-100% of the surface area of plant.

3. The plant of claim 2, comprising trichomes on 50-100% of the surface area of plant.

4. The plant of claim 2, comprising trichomes on 70-100% of the surface area of plant.

5. The plant of claim 1, comprising 20-80 mass % secondary compounds.

6. The plant of claim 5, comprising 30-70 mass % secondary compounds.

7. The plant of claim 5, comprising 40-65 mass % secondary compounds.

8. The plant of claim 5, comprising 30-60 mass % secondary compounds.

9. A plant of the genus cannabis comprising cDNA fragments corresponding to trichome induction in a plant not of genus cannabis.

10. The plant of claim 9, wherein the plant not of the genus cannabis is a plant of genus Arabidopsis.

11. A method of producing secondary compounds comprising inducing trichome development in a plant of the genus cannabis.

12. The method of claim 11, wherein said secondary compounds are chosen from cannabinoids or terpenes.

13. The method of claim 11, comprising harvesting at least one cannabinoid or at least one terpene during the vegetative growth cycle of the plant of the genus cannabis.

14. The method of claim 11, comprising modifying genetic material of the plant of the genus cannabis.

15. The method of claim 11, comprising introducing non-native DNA to the plant of the genus cannabis.

16. The method of claim 11, comprising introducing additional copies DNA native to the plant of the genus cannabis.

17. The method of claim 14, comprising overexpressing at least one trichome induction gene.

18. The method of claim 17, wherein the at least one trichome induction gene is chosen from the bHLH, WD40 repeat protein, R2R3-MYB, or R3-MYB families.

19. The method of claim 14, comprising infecting cells of the plant of genus cannabis with a transformed bacterium.

20. The method of claim 19, comprising infecting cells of the plant of genus cannabis with a transformed bacterium via syringe infiltration.

21. The method of claim 19, comprising infecting cells of the plant of genus cannabis with a transformed bacterium via vacuum infiltration.

22. The method of claim 14, comprising treating cells of the plant of genus cannabis with DNA attached to a metal particle via biolistic particle delivery.

23. The method of claim 14, comprising:
repressing post-transcriptional processing of a transcriptional repressor of trichome induction; and
repressing expression of a target gene as functional protein.

24. The method of claim 23, comprising:
expressing an RNA interference molecule corresponding to a trichome induction repression gene; and
reducing translation of mRNA trichome induction repression genes into functional protein.

25. The method of claim 23, comprising modifying expression of GLABROUS INFLORESENCE STEMS (GIS); GLABROUS INFLORESENCE STEMS 2 (G152); GLABORUS INFLORESCENCE STEMS 3 (GIS3)COTYLEDON TRICHOME 1 (COT1), MYB5, ARPC5, MYB106, FZR2, HOMEODOMAIN GLABROUS 12(HDG12), BRANCHLESS TRICHOMES (BTL), HOMEODOMAIN GLABROUS 11 (HDG11), STICHEL (STI), IRREGULAR TRICHOME BRANCH1 (ITB1), KAKTUS (KAK), MYOSIN XI K, ATXIK, DYNAMIN-RELATED PROTEIN (DRP1A), ATRLCK VI_A3, ZWICHEL, FRC1, FRC3, FRC4, ITB2, ITB3, ITB4, RASTIFARI (RFI), STA, SUZ1, SUZ2, SUZ3, ZINC FINGER PROTEIN 5, ZINC FINGRE PROTEIN 6, ZINC FINGER PROTEIN 8, GL2-EXPRESSION MODULATOR (GEM), HARLEQUIN (HQL), TRANSPARENT TESTA 8 (TT8) in a plant of genus cannabis.

26. The method of claim 23, comprising modifying expression of a gene chosen from the R2R3-MYB, bHLH, WD40 repeat protein, or R3-MYB gene families.

27. The method of claim 11, comprising physically altering the DNA of the plant of genus cannabis.
